(19) United States
(12) Patent Application Publication
(10) Pub. No.: US 2026/0047881 A1
(43) Pub. Date: Feb. 19, 2026

(54) MEDICAL INSTRUMENT KNIFE ASSEMBLY WITH BEAM AND OVERMOLD

(71) Applicant: Cilag GmbH International, Zug (CH)

(72) Inventors: Tyler N. Brehm, Arcanum, OH (US); Jason R. Lesko, Cincinnati, OH (US)

(21) Appl. No.: 18/804,250

(22) Filed: Aug. 14, 2024

(51) Int. Cl. A61B 18/14; A61B 18/00; A61B 34/30

(52) U.S. Cl. CPC A61B 18/1445; A61B 34/30; A61B 2018/0063; A61B 2018/1455

(57) ABSTRACT

An apparatus includes a body, a shaft assembly, and an end effector. The shaft assembly includes an outer shaft and a knife assembly. The knife assembly includes an elongate body and a beam. At least a portion of the body is slidably positioned within a hollow interior of the outer shaft. The beam extends distally from the distal end of the body. The beam includes a proximal portion positioned within the body and thereby secured to the body. The beam further includes a distal portion having a knife that is operable to sever tissue. The end effector includes a first jaw and a second jaw. The first jaw is movable relative to the second jaw between an open position and a closed position. The knife is operable to translate along one or both of the first jaw or the second jaw.

### BACKGROUND

A variety of surgical instruments include a tissue cutting element and one or more elements that transmit radio frequency (RF) energy to tissue (e.g., to coagulate or seal the tissue). Examples of such electrosurgical instruments and related concepts are disclosed in U.S. Pat. No. 6,500,176 entitled “Electrosurgical Systems and Techniques for Sealing Tissue,” issued Dec. 31, 2002, the disclosure of which is incorporated by reference herein, in its entirety; U.S. Pat. No. 9,526,565, entitled “Electrosurgical Devices,” issued Dec. 27, 2016, the disclosure of which is incorporated by reference herein, in its entirety; U.S. Pat. No. 11,857,247, entitled “Jaw for Surgical Instrument End Effector,” issued Jan. 2, 2024, the disclosure of which is incorporated by reference herein, in its entirety; and U.S. Pub. No. 2022/0008120, entitled “Electrosurgical Instrument with Floating Jaw Component,” published Jan. 13, 2022, the disclosure of which is incorporated by reference herein in its entirety.

An electrosurgical instrument may be powered by an external generator. Examples of such generators are disclosed in U.S. Pat. No. 8,986,302, entitled “Surgical Generator for Ultrasonic and Electrosurgical Devices,” issued Mar. 24, 2015, the disclosure of which is incorporated by reference herein, in its entirety.

Some electrosurgical instruments may be supported and driven by a robotic surgical system. Examples of robotically controlled electrosurgical instruments are disclosed in U.S. Pat. No. 11,576,738, entitled “Systems and Instruments for Tissue Sealing,” issued Feb. 14, 2023, the disclosure of which is incorporated by reference herein in its entirety; and U.S. Pub. No. 2022/0338891, entitled “Systems for Setting Jaw Gap in Surgical Tool End Effectors,” published Oct. 27, 2022, the disclosure of which is incorporated by reference herein in its entirety.

A knife assembly in an electrosurgical cutting instrument may need to have sufficient column strength to withstand longitudinal loads as a knife of the knife assembly is driven through tissue, without buckling or fracturing during the tissue cutting process. It may therefore be desirable to provide a substantially robust knife assembly. However, it may also be desirable to minimize the number of components of the knife assembly, simplify the process for manufacturing the knife assembly, and otherwise minimize the time and financial costs associated with the knife assembly. In some cases, the knife assembly of an electrosurgical cutting instrument may include one or more welds, which may complicate manufacturing and/or provide other undesirable results. It may therefore be desirable to provide a knife assembly in an electrosurgical cutting instrument that is substantially robust without providing the drawbacks associated with conventional knife assemblies.

While a variety of surgical instruments have been made and used, it is believed that no one prior to the inventors has made or used the invention described in the appended claims.

### BRIEF DESCRIPTION OF THE DRAWINGS

While the specification concludes with claims which particularly point out and distinctly claim this technology, it is believed this technology will be better understood from the following description of certain examples taken in conjunction with the accompanying drawings, in which like reference numerals identify the same elements.

FIG. 1 depicts a schematic view of an example of an electrosurgical instrument

FIG. 2A depicts a perspective view of an end effector and distal portion of a shaft assembly of the instrument of FIG. 1, with the end effector in a closed configuration.

FIG. 2B depicts a perspective view of the end effector and distal portion of the shaft assembly of FIG. 2A, with the end effector in an open configuration and a knife in a proximal position.

FIG. 2C depicts a perspective view of the end effector and distal portion of the shaft assembly of FIG. 2A, with the end effector in the open configuration and the knife in a distal position.

FIG. 3A depicts a side elevation view of the end effector and distal portion of the shaft assembly of FIG. 2A, with an outer shaft of the shaft assembly omitted, and with the end effector in the closed configuration.

FIG. 3B depicts a side elevation view of the end effector and distal portion of the shaft assembly of FIG. 2A, with the outer shaft of the shaft assembly omitted, and with the end effector in the open configuration.

FIG. 4 depicts a perspective view of a first jaw of the end effector of the instrument of FIG. 1.

FIG. 5 depicts a perspective view of a second jaw of the end effector of the instrument of FIG. 1.

FIG. 6 depicts an exploded perspective view of the shaft assembly of the instrument of FIG. 1.

FIG. 7 depicts an exploded perspective view of a distal portion of the shaft assembly of the instrument of FIG. 1.

FIG. 8 depicts a perspective view of a distal portion of a knife assembly of the shaft assembly of the instrument of FIG. 1.

FIG. 9 depicts a perspective cross-sectional view of the distal portion of the knife assembly of FIG. 8, with the cross-section taken along a longitudinally plane laterally bisecting the knife assembly.

FIG. 10 depicts another perspective view of the distal portion of the knife assembly of FIG. 8.

FIG. 11 depicts a perspective view of the distal portion of an inner guide of the shaft assembly of the instrument of FIG. 1.

FIG. 12A depicts a perspective view of intermediate portions of the knife assembly of FIG. 8 and the inner guide of FIG. 11, with the knife assembly in a first longitudinal position relative to the inner guide.

FIG. 12B depicts a perspective view of intermediate portions of the knife assembly of FIG. 8 and the inner guide of FIG. 11, with the knife assembly in a second longitudinal position relative to the inner guide.

FIG. 13 depicts a perspective view of a proximal portion of the knife assembly of FIG. 8.

FIG. 14 depicts a perspective view of a proximal portion of the shaft assembly of the instrument of FIG. 1, with an exploded perspective view of a knife firing assembly of the instrument of FIG. 1.

FIG. 15 depicts an exploded perspective view of a clip and retainer of the knife firing assembly of FIG. 14.

FIG. 16 depicts a cross-sectional view of the proximal portion of the shaft assembly and the knife firing assembly of FIG. 14, taken along line 16-16 of FIG. 17A.

FIG. 17A depicts a perspective view of the proximal portion of the shaft assembly and the knife firing assembly of FIG. 14, with the knife firing assembly in a first longitudinal position relative to the shaft assembly.

FIG. 17B depicts a perspective view of the proximal portion of the shaft assembly and the knife firing assembly of FIG. 14, with the knife firing assembly in a second longitudinal position relative to the shaft assembly.

### DETAILED DESCRIPTION

Any one or more of the teachings, expressions, embodiments, examples, etc. described herein may be combined with any one or more of the other teachings, expressions, embodiments, examples, etc. that are described herein. The following-described teachings, expressions, embodiments, examples, etc. should therefore not be viewed in isolation relative to each other. Various suitable ways in which the teachings herein may be combined will be readily apparent to those skilled in the art in view of the teachings herein. Such modifications and variations are intended to be included within the scope of the claims.

### I. Overview of Electrosurgical Instrument

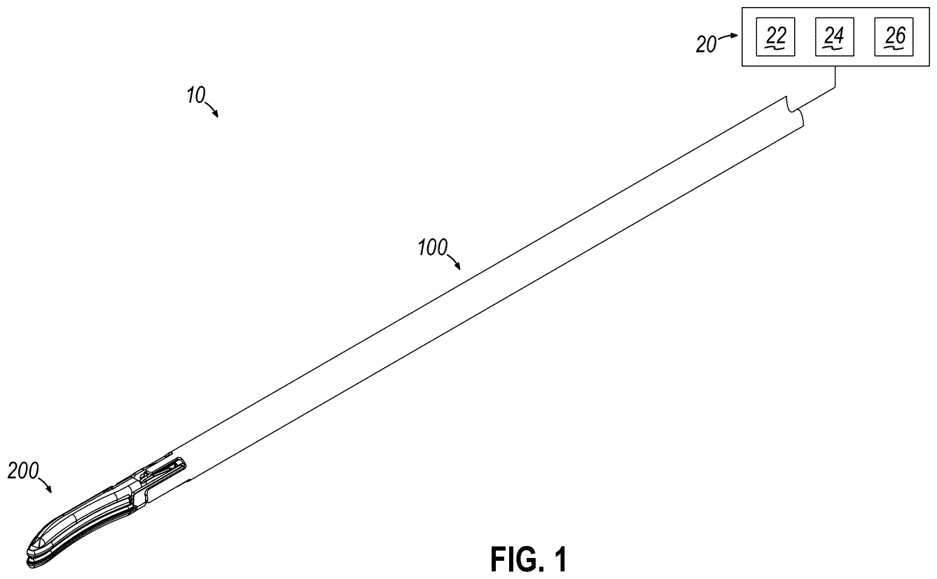

FIG. 1 shows an example of an electrosurgical instrument (10) that may be used in numerous kinds of medical procedures. Instrument (10) of this example includes a body assembly (20), a shaft assembly (100), and an end effector (200). Shaft assembly (100) extends distally relative to body assembly (20). End effector (200) is positioned at a distal end of shaft assembly (100). As will be described in greater detail below, end effector (200) is operable to grasp, cut, and seal or weld tissue (e.g., a blood vessel, etc.). Specifically, end effector (200) is operable to grasp tissue between opposing jaws (220, 240), cut the tissue with a knife (184), and seal or weld the tissue by applying bipolar RF energy to the tissue via respective electrodes (224, 244) of jaws (220, 240). While not shown in FIG. 1, instrument (10) may be coupled with a generator that is operable to provide the RF energy to electrodes (224, 244).

Body assembly (20) may take numerous different forms. In some versions, body assembly (20) includes a handle, such that body assembly (20) is configured to be grasped and manipulated by at least one hand of a human operator. By way of example only, such a handle may include a pistol grip, a scissor grip, or any other suitable configuration. In some other versions, body assembly (20) is configured to be coupled with a robotic arm or other component of a robotic surgical system, such that body assembly (20) is configured to be supported and activated by the robotic surgical system. Alternatively, body assembly (20) may take any other suitable form.

Body assembly (20) of the present example includes a jaw closure assembly (22), a knife drive assembly (24), and an input interface (26). Jaw closure assembly (22) is operable to drive jaws (220, 240) of end effector between an open position and a closed position, with closure motion from jaw closure assembly (22) being communicated to end effector (200) via shaft assembly (100) as will be described in greater detail below. Any suitable components and arrangements may be used to form jaw closure assembly (22) as will be apparent to those skilled in the art in view of the teachings herein. Knife drive assembly (24) is operable to drive knife (184) between a proximal position and a distal position, with translational motion from knife drive assembly (24) being communicated to end effector (200) via shaft assembly (100) as will be described in greater detail below. Any suitable components and arrangements may be used to form knife drive assembly (24) as will be apparent to those skilled in the art in view of the teachings herein.

Input interface (26) is operable to interface with a human or robotic operator to activate jaw closure assembly (22), knife drive assembly (24), and/or communication of RF energy to electrodes (224, 244). In versions where body assembly (30) is configured to be grasped and manipulated by at least one hand of a human operator, input interface (26) may include one or more trigger, one or more levers, one or more sliders, one or more buttons, and/or any other suitable kind(s) of input features, including combinations thereof. In versions where body assembly (30) is configured to be coupled with a robotic arm or other component of a robotic surgical system, input interface (26) may include one or more gears, one or more drive spindles, one or more shuttles or other translating drive elements, and/or other kind(s) of features that mechanically interface with complementary features of the robotic arm or other component of the robotic surgical system. In addition, or in the alternative, in versions where body assembly (30) is configured to be coupled with a robotic arm or other component of a robotic surgical system, input interface (26) may include one or more pins or sockets, one or more other kinds of electrical contacts, and/or any other kind(s) of features that electrically interface with complementary features of the robotic arm or other component of the robotic surgical system.

### A. Overview of End Effector

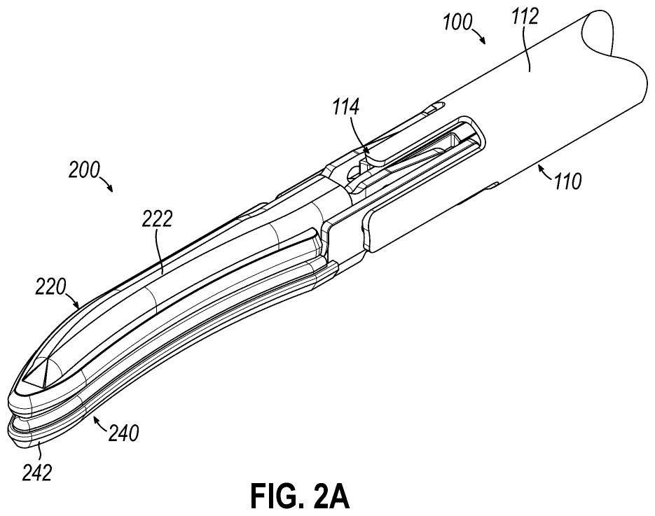

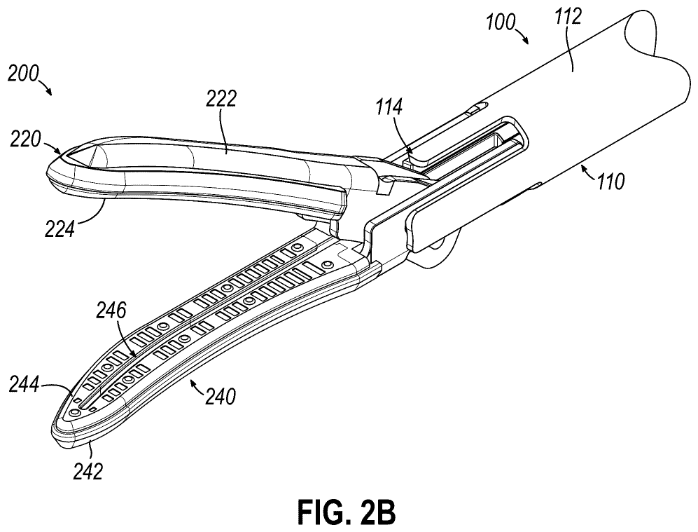

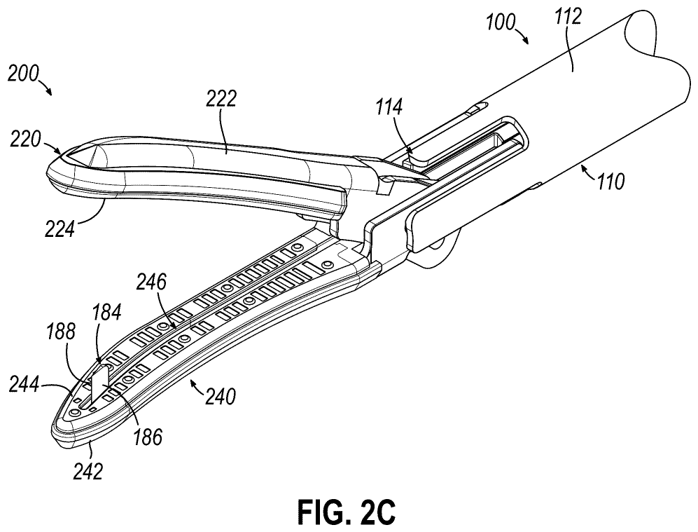

As shown in FIGS. 2A-2C, and as noted above, end effector (200) of the present example includes a first jaw (220) and a second jaw (240) that are operable to transition between a closed configuration (FIG. 2A) and an open configuration (FIG. 2B). In the present example, jaw (220) pivots toward and away from jaw (240), while jaw (240) remains stationary relative to shaft assembly (100), as jaws (220, 240) transition between the open configuration and the closed configuration. In some other versions, both jaws (220, 240) pivot simultaneously toward and away from each other in opposing fashion to transition between the open configuration and the closed configuration. Alternatively, one or both of jaws (220, 240) may move in any other suitable fashion.

As shown in the transition from FIG. 2B to FIG. 2C, knife (184) is operable to translate relative to jaws (220, 240) from a proximal position (FIG. 2B) to a distal position (FIG. 2C). While knife (184) is shown as being in the distal position while jaws (220, 240) are in the open configuration, some versions of operation may provide translation of knife (184) from the proximal position to the distal position only when jaws (220, 240) are in the closed configuration. In other words, in some versions of instrument (10) and methods of use of instrument (10), knife (184) would not be in the distal position when jaws (220, 240) are in the open configuration. The arrangement shown in FIG. 2C should therefore be understood as only being provided for purposes of illustration to show a distal position of knife (184) in relation to jaw (240).

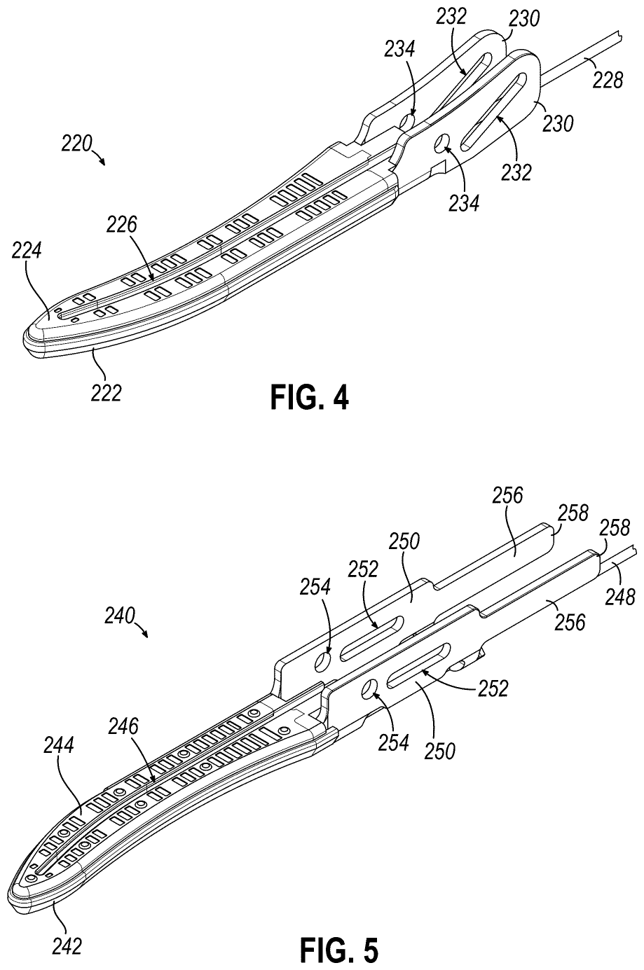

As shown in FIGS. 2A-4 jaw (220) includes a body (222) and an electrode (224). In versions where body (222) includes an electrically conductive material (e.g., metal, etc.), an electrically insulative material (e.g., polymer, etc.) may be interposed between body (222) and electrode (224) to electrically isolate the electrically conductive portion of body (222) relative to electrode (224). For instance, an insulative plastic material may be overmolded over a metallic portion of body (222) (or otherwise be secured to the metallic portion of body (222)), and electrode (224) may be secured to the other side of the plastic material, such that the plastic material is interposed between electrode (224) and the metallic portion of body (222). In the present example, body (222) and electrode (224) curve laterally away from a central longitudinal axis along a distal portion of the length of jaw (220). In some other versions, these features are substantially straight along the entire length of jaw (220).

Jaw (220) defines a knife slot (226) passing through electrode (224). Knife slot (226) is configured to accommodate longitudinal motion of knife (184) relative to jaw (220) while jaws (220, 240) are in the closed configuration. Jaw (220) further includes a pair of proximal flanges (230) which are laterally spaced apart from each other. Each flange (230) defines an elongate slot (232) and a pin opening (234). Each elongate slot (232) is oriented obliquely along its corresponding flange (230), which provides closure motion to jaw (220) as described in greater detail below. A wire (228) extends proximally from jaw (220). Wire (228) is coupled with electrode (224) and extends along the length of shaft assembly (100) as described in greater detail below. Wire (228) is further configured to couple with a source of RF energy (e.g., an external generator), such that wire (228) is configured to provide RF energy to electrode (224).

As shown in FIGS. 2A-3 and 5 jaw (240) includes a body (242) and an electrode (244). In versions where body (242) includes an electrically conductive material (e.g., metal, etc.), an electrically insulative material (e.g., polymer, etc.) may be interposed between body (242) and electrode (244) to electrically isolate the electrically conductive portion of body (242) relative to electrode (244). For instance, an insulative plastic material may be overmolded over a metallic portion of body (242) (or otherwise be secured to the metallic portion of body (242)), and electrode (244) may be secured to the other side of the plastic material, such that the plastic material is interposed between electrode (244) and the metallic portion of body (242). In the present example, body (242) and electrode (244) curve laterally away from a central longitudinal axis along a distal portion of the length of jaw (240). In some other versions, these features are substantially straight along the entire length of jaw (240).

Jaw (240) defines a knife slot (246) passing through electrode (244). Knife slot (246) is configured to accommodate longitudinal motion of knife (144) relative to jaw (240). Jaw (240) further includes a pair of proximal flanges (250) which are laterally spaced apart from each other. Each flange (250) defines an elongate slot (252) and a pin opening (254). Each elongate slot (252) is oriented longitudinally along its corresponding flange (250). A beam (256) extends proximally from each flange (250) and terminates in a respective proximal face (258). A wire (248) extends proximally from jaw (240). Wire (248) is coupled with electrode (244) and extends along the length of shaft assembly (100) as described in greater detail below. Wire (248) is further configured to couple with a source of RF energy (e.g., an external generator), such that wire (248) is configured to provide RF energy to electrode (244).

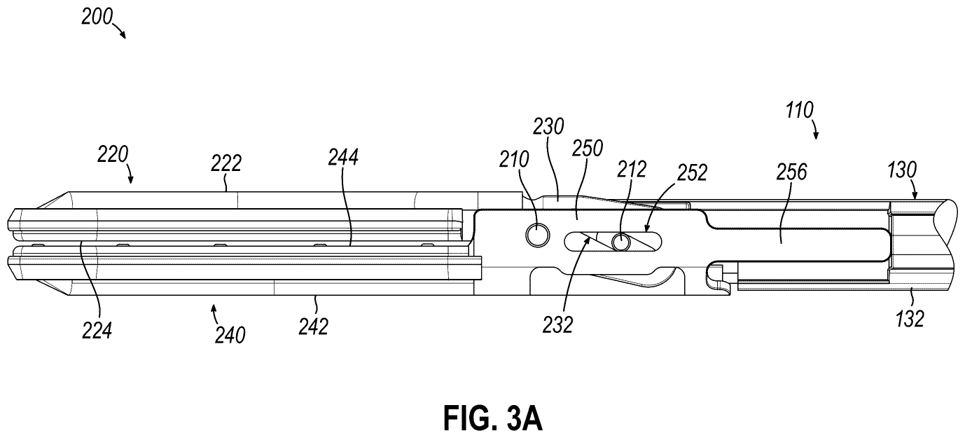

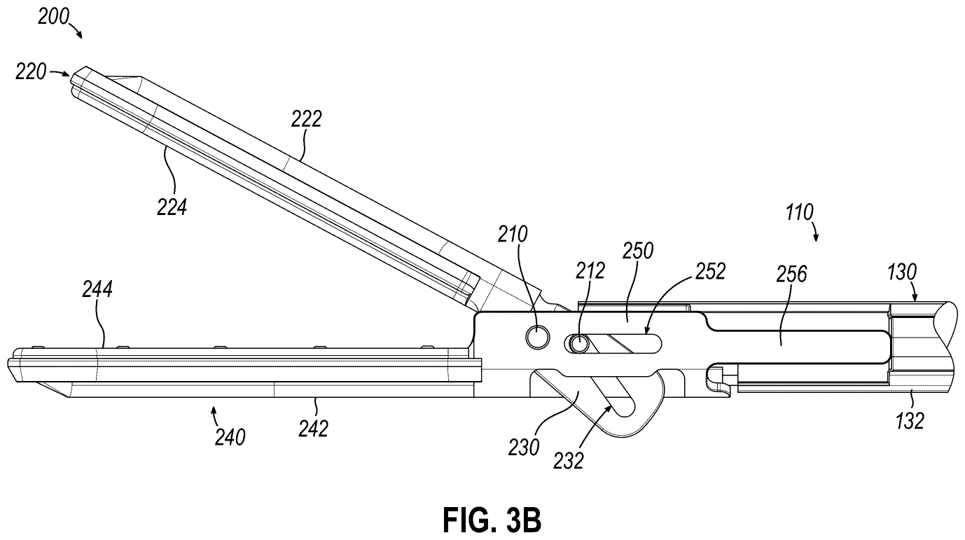

As shown in FIGS. 3A-3B, a pin (210) is disposed through openings (234, 254) of flanges (230, 250) of jaws (220, 240), thereby pivotably coupling jaws (220, 240) to each other. Another pin (212) is disposed through slots (232, 252). Pin (212) is operable to be driven between a proximal position (FIG. 3A) and a distal position (FIG. 3B) as will be described in greater detail below. Because slots (232) of flanges (230) are oriented obliquely while slots (252) of flanges (250) are oriented longitudinally in this example, longitudinal motion of pin (212) along slots (232, 252) will drive pivotal motion of jaws (220, 240). In particular, pin (212) will drive jaw (220) to an open position when pin (212) is in a proximal position (FIG. 3A); and to a closed position when pin (212) is in a distal position (FIG. 3B). Alternatively, jaws (220, 240) may be driven between open and closed configurations using any other suitable features or techniques.

In use, tissue (e.g., a vessel, etc.) may be positioned between jaws (220, 240) while jaws (220, 240) are in an open configuration. Then, jaws (220, 240) may be driven to the closed configuration to clamp the tissue. While jaws (220, 240) remain closed, knife (184) may be driven from the proximal position to the distal position to sever the tissue. In addition, bipolar RF energy may be applied to the tissue via electrodes (224, 244) to thereby seal the tissue. In some versions, electrode (244) serves as an active electrode while electrode (224) serves as a return electrode. Alternatively, this relationship may be reversed. In some examples of use, bipolar RF energy may be applied to the tissue via electrodes (224, 226) without knife (184) being advanced distally through the tissue. In either scenario, jaws (220, 240) may be returned to the open configuration to release the tissue when appropriate.

### B. Overview of Shaft Assembly

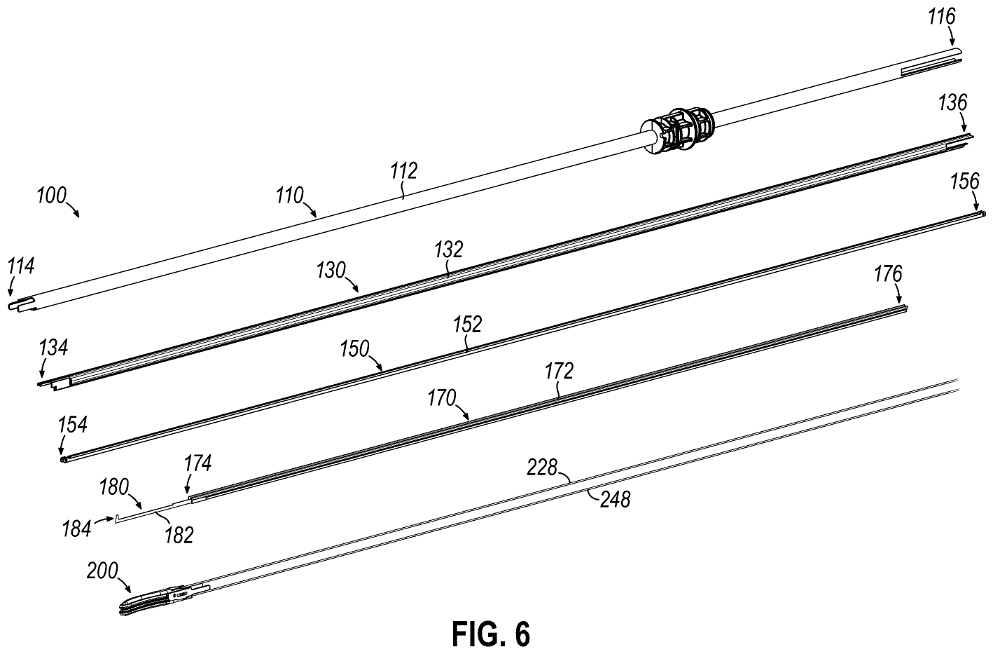

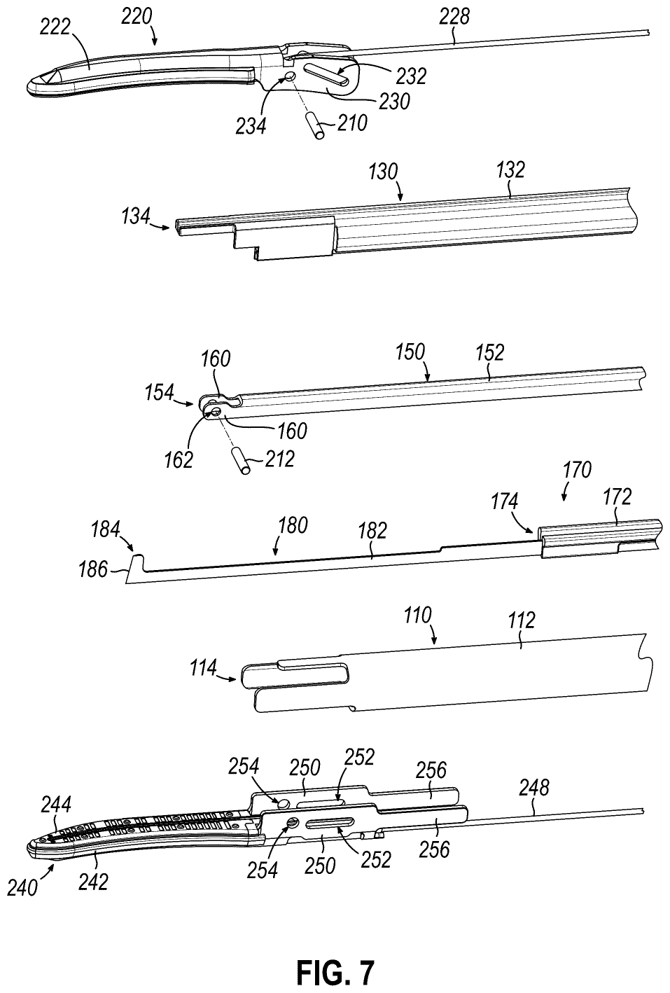

FIGS. 6-7 show components of shaft assembly (100) in greater detail. As shown, shaft assembly (100) of this example includes an outer shaft (110), an inner guide (130), a closure beam (150), and a knife assembly (170). Outer shaft (110) of this example includes a hollow tubular body (112) with a distal end (114) and a proximal end (116). End effector (200) is positioned at distal end (114) while proximal end (116) is disposed in body (20). Inner guide (130) of this example includes an elongate body (132) with a distal end (134) and a proximal end (136). Elongate body (132) of inner guide (130) is disposed within tubular body (112) of outer shaft (110). Elongate body (132) is laterally offset relative to the central longitudinal axis of tubular body (112) (i.e., not centered along the central longitudinal axis of tubular body (112)). Elongate body (132) of inner guide (130) remains stationary relative to tubular body (112) of outer shaft (110) during operation of instrument (10) in the present example. End effector (200) is positioned at distal end (134) while proximal end (136) is disposed in body (20). Wires (228, 248) are interposed between elongate body (132) of inner guide (130) and the inner surface of tubular body (112) of outer shaft (110) in this example.

Closure beam (150) of this example includes an elongate body (152) with a distal end (154) and a proximal end (156). Elongate body (152) of closure beam (150) is disposed within tubular body (112) of outer shaft (110), laterally adjacent to elongate body (132) of inner guide (130). In the present example, elongate body (152) is radially centered along the central longitudinal axis of tubular body (112). Elongate body (152) of the present example has an upside-down “U” shaped cross-sectional profile, though elongate body (152) may alternatively have any other suitable cross-sectional profile. Proximal end (156) is disposed within body (20) and is coupled with jaw closure assembly (22), such that jaw closure assembly (22) is operable to drive longitudinal translation of closure beam (150) relative to outer shaft (110) and relative to inner guide (130). Distal end (154) includes a clevis arrangement formed by two flanges (160), each flange (160) having a respective pin opening (162). Pin (212) is disposed in openings (162), in addition to being disposed in slots (232, 252) of flanges (230, 250) of jaws (220, 240). Thus, closure beam (150) drives translation of pin (212) to thereby drive opening and closing of jaws (220, 240) as described above. Alternatively, any other suitable components and arrangements may be used to drive opening and closing of jaws (220, 240).

Knife assembly (170) of the present example includes an elongate body (172) and a knife beam (180). Knife beam (180) is fixedly secured relative to elongate body (172) and extends distally relative to a distal end (174) of elongate body (172). Knife beam (180) includes a beam body (182) that distally terminates in knife (184). Knife (184) includes an upright portion (186) having a distal cutting edge (188). Elongate body (172) of knife assembly (170) is disposed within tubular body (112) of outer shaft (110), laterally adjacent to elongate body (132) of inner guide (130) and elongate body (152) of closure beam (150). Elongate body (172) is laterally offset relative to the central longitudinal axis of tubular body (112) (i.e., not centered along the central longitudinal axis of tubular body (112)), though the central longitudinal axis of tubular body (112) extends along a portion of elongate body (172) in this example. Elongate body (172) of the present example has a “U” shaped cross-sectional profile, though elongate body (152) may alternatively have any other suitable cross-sectional profile. In the present example, the “U” shape of elongate body (172) is partially nested within the “U” shape of elongate body (152).

A proximal end (176) of elongate body (172) is disposed within body (20) and is coupled with knife drive assembly (24), such that knife drive assembly (24) is operable to drive longitudinal translation of knife assembly (170) relative to outer shaft (110) and relative to inner guide (130). In the present example, knife assembly (170) and closure beam (150) are translatable independently relative to each other. Also in the present example, knife assembly (170) and knife drive assembly (24) are configured such that when knife (184) is at the distal-most position of the range of motion of knife (184) cutting edge (188) is spaced proximally from the distal ends of knife slots (226, 246). In other words, cutting edge (188) will not contact jaws (220, 240) at the distal ends of knife slots (226, 246) when knife (184) reaches the distal-most position of the range of motion of knife (184). This may prevent premature dulling of cutting edge (188) that might otherwise occur if cutting edge (188) were allowed to contact jaws (220, 240) at the distal ends of knife slots (226, 246) when knife (184) reaches the distal-most position of the range of motion of knife (184). In some other versions, knife assembly (170) and knife drive assembly (24) are configured to allow cutting edge (188) to contact jaws (220, 240) at the distal ends of knife slots (226, 246) when knife (184) reaches the distal-most position of the range of motion of knife (184).

### II. Example Features of Knife Assembly

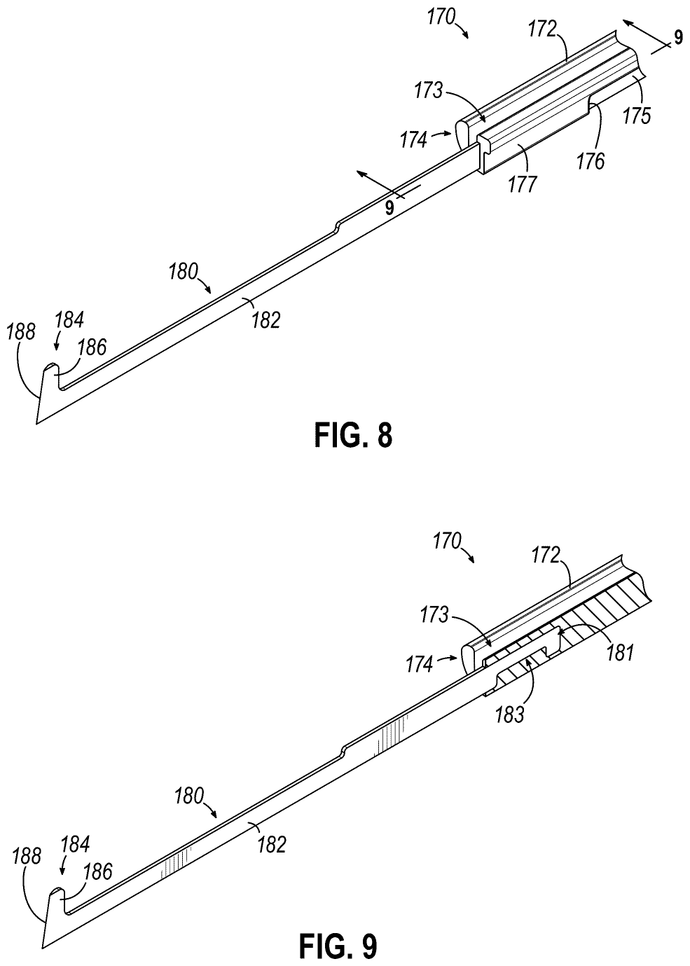

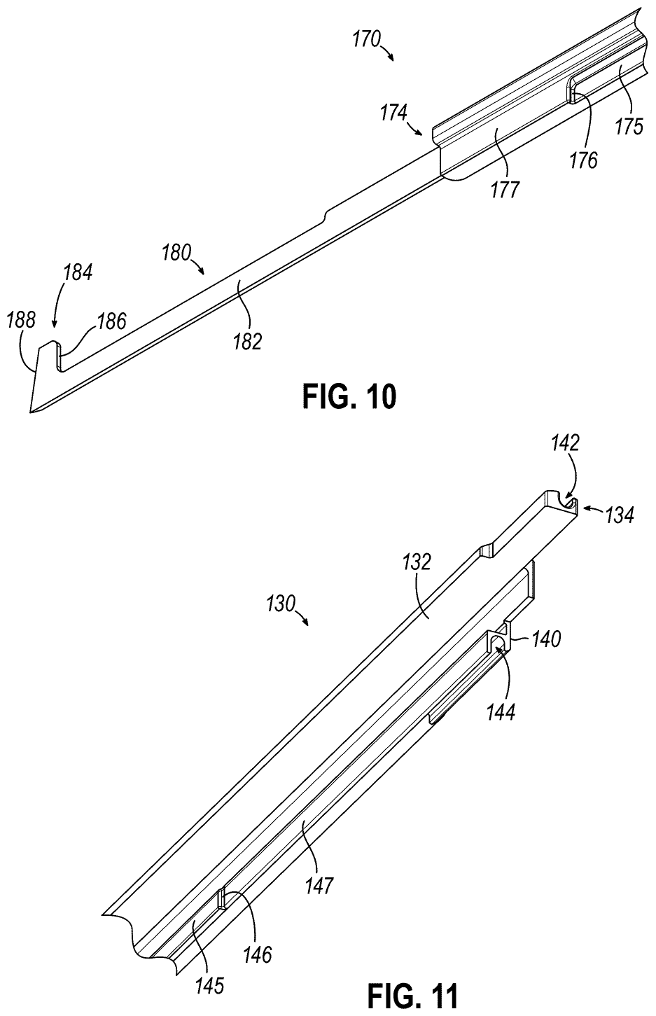

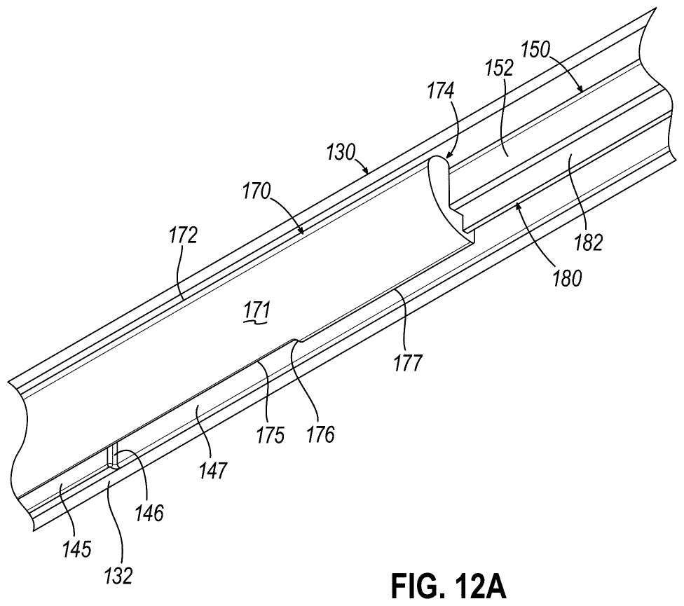

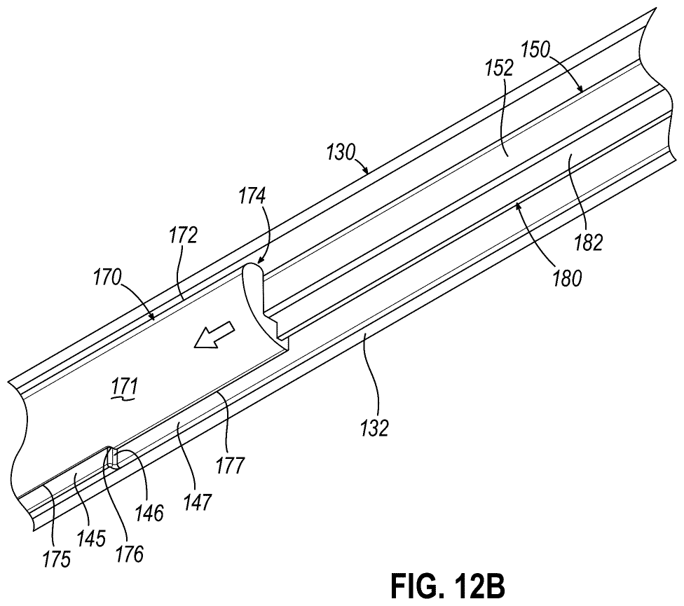

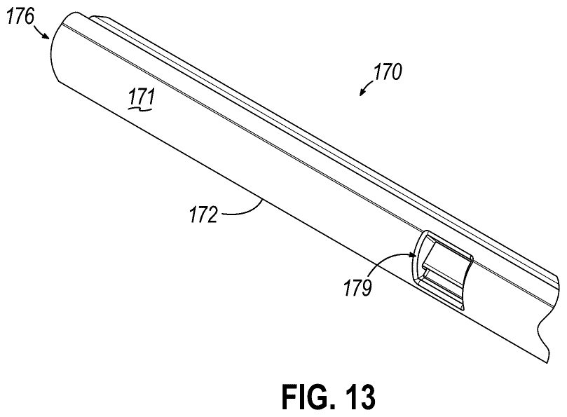

As noted above, it may be desirable to provide a substantially robust knife assembly in an electrosurgical instrument while also minimizing the number of components of the knife assembly, simplify the process for manufacturing the knife assembly, and otherwise minimize the time and financial costs associated with the knife assembly. Knife assembly (170) of the present example may provide at least some of these benefits and functionalities. As noted above, knife assembly (170) includes an elongate body (172) and a knife beam (180). These features are shown in greater detail in FIGS. 8-10. As shown, elongate body (172) includes a curved outer surface (171) (best seen in FIGS. 12A-13, an inwardly extending recess (173) or channel, a first laterally facing surface (175), a second laterally facing surface (177), and a proximally facing surface (176) that provides a transition between surfaces (175, 177).

In the present example, the central longitudinal axis of elongate body (172) is laterally offset relative to the centra longitudinal axis of tubular body (112) of outer shaft (110). Curved outer surface (171) is contoured to complement the contour of inner surface of tubular body (112). In some versions, curved outer surface (171) maintains sliding contact with the inner surface of tubular body (112) along the entire area of curved outer surface (171). Such sliding contact may contribute to the robustness of shaft assembly (100) (e.g., preventing buckling of elongate body (172) during distal advancement of knife assembly (170), etc.). Surface (177) extends to distal end (174) of elongate body (172). Surface (175) is inwardly recessed relative to surface (177), such that elongate body (172) has greater lateral thickness along the longitudinal region of surface (177) than along the longitudinal region of surface (175).

Recess (173) is configured to slidably receive a portion of elongate body (152) of closure beam (150), such that elongate body (172) of knife assembly (170) and elongate body (152) of closure beam (150) are in a nested arrangement in this example. This nested arrangement may prevent buckling of elongate body (172) and/or closure beam (150) during operation of instrument (10), such that elongate bodies (152, 172) provide structural support to each other while still allowing closure beam (150) and knife assembly (170) to translate longitudinally relative to each other.

As noted above, the distal portion of knife beam (180) includes a beam body (182) with a knife (184), which includes an upright portion (186) having a distal cutting edge (188). In some versions, knife beam (180) is formed as stamped metal blank, though other suitable processes may be used. Knife (184) may be formed through any suitable process or processes, including but not limited to grinding, stamping, etching, etc. In the present example, elongate body (172) comprises a plastic material that is overmolded about a proximal region of beam body (182). By forming elongate body (172) as an overmold, this may facilitate the optimization of the shape of elongate body (172) based on finite element analysis (FEA) and size requirements, etc., where such optimization may be impractical or impossible using some other methods of manufacturing elongate body (172).

To facilitate retention of knife beam (180) relative to elongate body (172), beam body (182) includes an elongate notch (183) formed just distal to proximal end (181) of beam body (182), as best seen in FIG. 9. This notch (183) receives plastic material of elongate body (172) during the overmolding process, which provides a secure, rigid fit between elongate body (172) and knife beam (180). In some other versions, beam body (182) has a plurality of openings, ripples, and/or other features near proximal end (181) to promote retention of beam body (182) relative to elongate body (172).

As noted above, elongate body (172) has greater lateral thickness along the longitudinal region of surface (177) than along the longitudinal region of surface (175). This region of greater lateral thickness of elongate body (172) corresponds with the longitudinal region where the material of elongate body (172) is disposed in notch (183), such that the greater lateral thickness contributes to the robustness of the securement between elongate body (172) and knife beam (180).

In the present example, no welding steps are used to form knife assembly (170). The above-described method for manufacturing knife assembly (170) may provide minimal cost of time and other resources. Moreover, the combination of knife beam (180) and elongate body (172) may still provide substantial robustness to knife assembly (170). Elongate body (172) and knife beam (180) together provide sufficient column strength to withstand longitudinal loads as knife (184) is driven through tissue, particularly in view of lateral structural support provided by closure beam (150) and inner guide (130). It should be understood that other manufacturing methods may be used to form knife assembly (170), though some other manufacturing methods may undesirably increase cost of time and/or other resources.

As noted above, closure beam (150) may provide lateral support to knife assembly (170) with a portion of closure beam (150) being slidably nested within recess (173) of elongate body (172). Inner guide (130) may also provide lateral support to knife assembly (170). FIG. 11 shows inner guide (130) in further detail. As noted above, inner guide (130) includes an elongate body (132). As shown in FIG. 11, elongate body (132) of inner guide (130) includes a first channel (142) and a second channel (144), with each channel (142, 144) extending along the full length of elongate body (132). Channel (142) is sized and positioned to receive wire (228) while channel (144) is sized and positioned to receive wire (248). Elongate body (132) further includes a flat, outwardly laterally facing surface (140) near distal end (134), a first inwardly laterally facing surface (145), a second inwardly laterally facing surface (147), and a distally facing surface (146) that provides a transition between surfaces (145, 147). Surface (147) is positioned distally in relation to surface (145). Surface (145) is inwardly recessed relative to surface (147), such that elongate body (132) has greater lateral thickness along the longitudinal region of surface (145) than along the longitudinal region of surface (145).

FIGS. 12A-12B show inner guide (130) and knife assembly (170) together at different stages of operation of instrument (10)—namely, when knife assembly (170) is in a distal position (FIG. 12A) and when knife assembly (170) is in a proximal position (FIG. 12B). As shown, surface (177) of elongate body (172) is positioned to face and slide along surface (147) of elongate body (132), while surface (175) of elongate body (172) is positioned to face and slide along surface (145) of elongate body (132). Surfaces (146, 176) are similarly dimensioned along the transverse plane, such that the lateral offset between surfaces (145, 147) is substantially the same as the lateral offset between surfaces (175, 177). The lateral offset provided by surfaces (146, 147) thus allows elongate body (132) of inner guide (130) to accommodate the increased thickness of elongate body (172) along the length of surface (177). Similarly, the lateral offset provided by surfaces (146, 145) allows elongate body (132) of inner guide (130) to make up for the reduced thickness of elongate body (172) along the length of surface (175).

In the example shown in FIG. 12B, surface (176) is positioned substantially adjacent to surface (146) when knife assembly (170) is in the proximal position. In some versions, surface (176) contacts surface (146) when knife assembly (170) is in the proximal position. In some other versions, surface (176) is longitudinally spaced away from surface (146) when knife assembly (170) is in the proximal position, such that a longitudinally extending gap is defined between surfaces (146, 176) when knife assembly (170) is in the proximal position. In some such versions, and as will be described in greater detail below, clip (330) of knife firing assembly (300) contacts the proximal end of elongate opening (118) of tubular body (112) when knife assembly (170) is in the proximal position. Such contact may provide a hard stop to the proximal positioning of knife assembly (170).

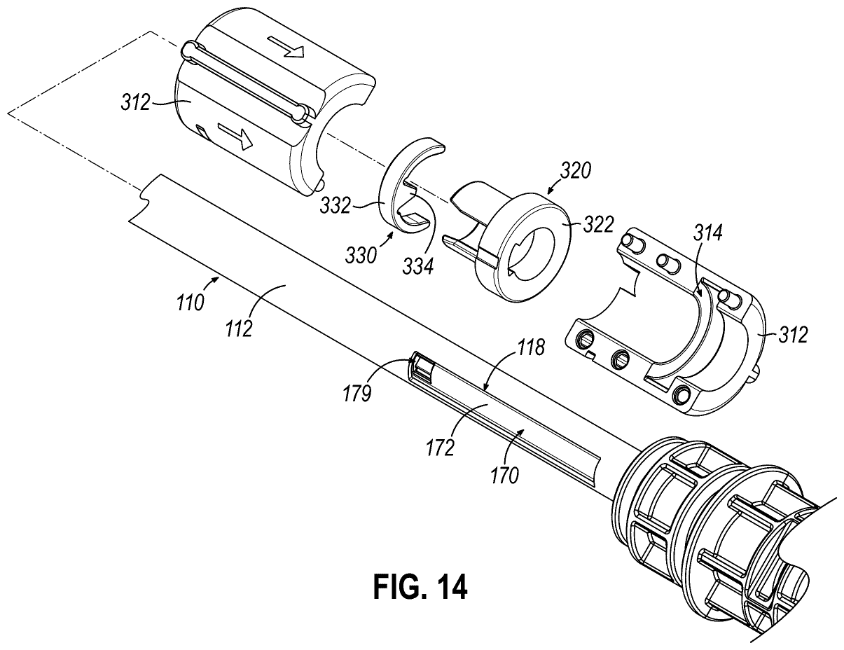

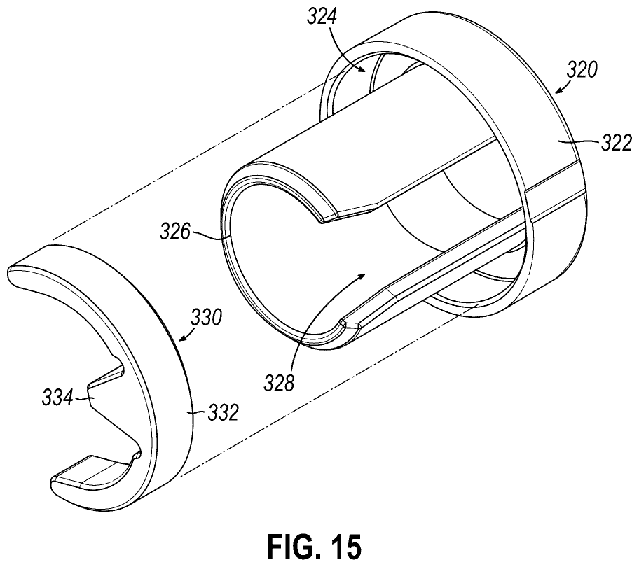

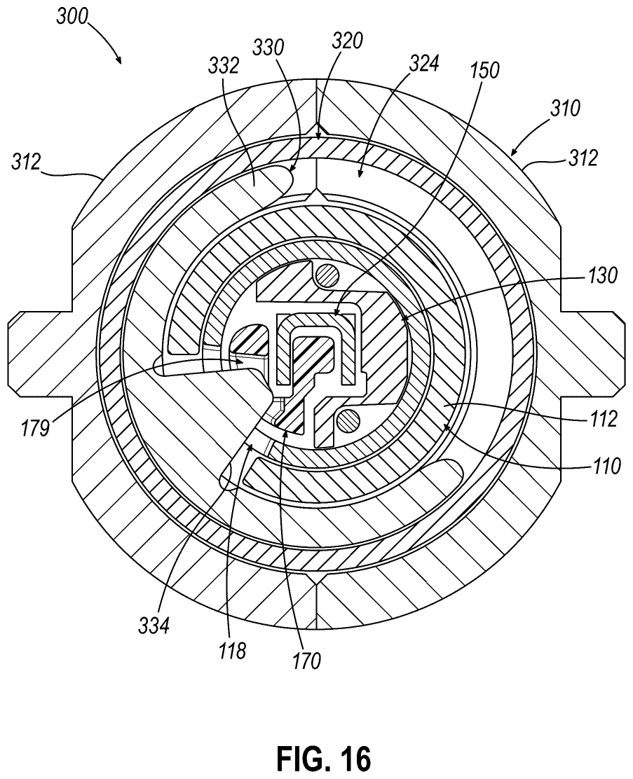

FIGS. 13-17B show an example of components of a knife firing assembly (300) and features of knife assembly (170) that interact with knife firing assembly (300). As shown in FIG. 13, elongate body (172) of knife assembly (170) defines an inwardly formed notch (179) near (but distally spaced from) proximal end (176) of elongate body (172). As shown in FIGS. 14-15, knife firing assembly (300) of this example includes a collar assembly (310), a retainer (320), and a clip (330).

Collar assembly (310) may couple with knife drive assembly (24) in any suitable fashion, such that knife drive assembly (24) may drive longitudinal translation of knife assembly (170) by driving longitudinal translation of collar assembly (310). Collar assembly (310) of the present example includes a pair of collar bodies (312) that are fixedly joined together to encompass retainer (320) and clip (330) about a proximal portion of shaft assembly (100). As shown in FIG. 14, each collar body (312) defines an internal recess (314). When collar bodies (312) are coupled together, recesses (314) cooperate to form a continuous annular recess (314).

As shown in FIGS. 14-15, retainer (320) of the present example includes an annular flange (322) and a cylindraceous body (326) extending distally from annular flange (322). Flange (322) is configured to fit in recesses (314) of collar assembly (310), such that longitudinal movement of collar assembly (310) will provide longitudinal movement of retainer (320). As best seen in FIG. 15, flange (322) defines a distally-facing, proximally-extending annular recess (324). As also best seen in FIG. 15, body (326) defines a longitudinally extending notch (328).

As shown in FIGS. 14-16, clip (330) of the present example includes an arcuate body (332) with an inwardly protruding tab (334). Body (332) is configured to fit within recess (324) of flange (322). Notch (328) of body (326) is configured to accommodate tab (334), such that tab (334) protrudes through tab (334). In the present example, tab (334) is disposed in notch (179) of elongate body (172) of knife assembly (170). This fit between tab (334) and notch (179) is sufficiently close to allow clip (330) to directly drive movement of knife assembly (170) without substantial play of tab (334) within notch (179). It should be understood that retainer (320) and collar assembly (310) may substantially secure clip (330) relative to knife assembly (170), thereby maintaining the position of tab (334) in notch (179) throughout operation of instrument (10).

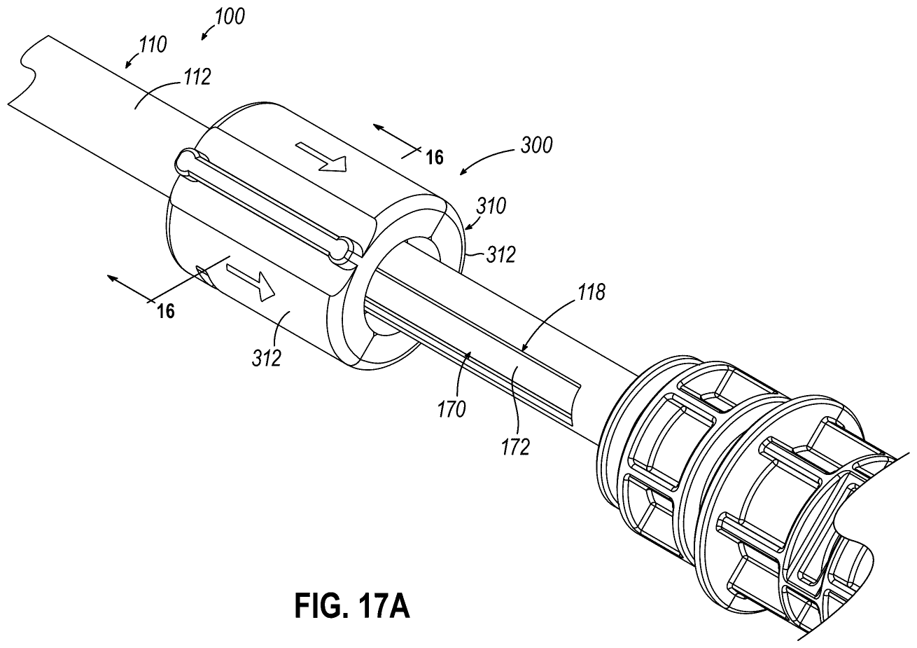

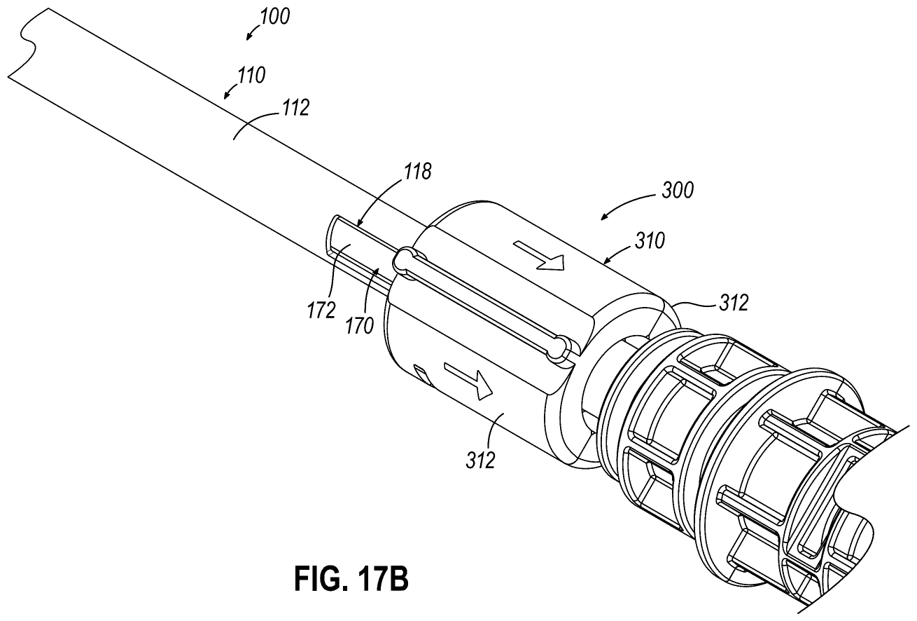

FIGS. 17A and 17B show knife firing assembly (300) being used to drive knife assembly (170) between a distal position (FIG. 17A) and a proximal position (FIG. 17B. As shown in FIGS. 14 and 17A-17B, an elongate opening (118) is formed along a proximal portion of tubular body (112) of outer shaft (110). Opening (118) is positioned to expose notch (179) and has a length that is at least as long as the range of longitudinal motion of knife assembly (170). Thus, when knife firing assembly (300) is fully assembled, tab (334) protrudes through opening (118) to enter notch (179). As knife assembly (170) transitions between the distal position (FIG. 17A) and the proximal position (FIG. 17B), tab (334) travels along at least part of the length of opening (118).

In some versions, the length of opening (118) is greater than the length of the range of longitudinal motion of knife assembly (170). In some versions, tab (334) engages the distal-most edge of opening (118) to thereby restrict the distal advancement of knife assembly (170). Alternatively, engagement between the proximal end of a beam (256) of jaw (240) and distal end (174) of elongate body (172) may restrict the distal advancement of knife assembly (170). In either case, providing a hard stop restricting the distal advancement of knife assembly (170) may prevent distal cutting edge (188) of knife (184) from contacting one or both of jaws (220, 240) at the distal end(s) of either or both of knife slots (226, 246). This may in turn prevent premature dulling of cutting edge (188) or damage to cutting edge (188). In addition, or in the alternative, tab (334) may engage the proximal-most edge of opening (118) to thereby restrict the proximal retraction of knife assembly (170).

### III. Examples of Combinations

The following examples relate to various non-exhaustive ways in which the teachings herein may be combined or applied. The following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### Example 1

An apparatus comprising: (a) a body; (b) a shaft assembly extending distally relative to the body, the shaft assembly including: (i) an outer shaft defining a hollow interior, and (ii) a knife assembly including: (A) an elongate body having a proximal end and a distal end, at least a portion of the body being slidably positioned within the hollow interior of the outer shaft, and (B) a beam extending distally from the distal end of the body, the beam including a proximal portion positioned within the body and thereby secured to the body, the beam further including a distal portion having a knife, the knife being operable to sever tissue; and (c) an end effector positioned at a distal end of the shaft assembly, the end effector including: (i) a first jaw, and (ii) a second jaw, the first jaw being movable relative to the second jaw between an open position and a closed position, the knife being operable to translate along one or both of the first jaw or the second jaw.

### Example 2

The apparatus of Example 1, the body comprising a plastic material.

### Example 3

The apparatus of Example 2, the beam comprising a metal material.

### Example 4

The apparatus of Example 3, the plastic material in a distal portion of the body surrounding the metal material in the proximal portion of the beam as an overmold about the metal material.

### Example 5

The apparatus of any of Examples 1 through 4, the body having a length, the body having a first thickness along a first region of the length of the body, the body having a second thickness along a second region of the length of the body, the proximal portion of the beam being disposed within the second region of the length of the body.

### Example 6

The apparatus of any of Examples 1 through 5, the proximal portion of the beam including a notch, the notch being configured to promote a rigid coupling between the beam and the body.

### Example 7

The apparatus of any of Examples 1 through 6, the outer shaft defining a central longitudinal axis, the body of the knife assembly defining a central longitudinal axis, the central longitudinal axis of the knife assembly being laterally offset relative to the central longitudinal axis of the outer shaft.

### Example 8

The apparatus of any of Examples 1 through 7, the shaft assembly further including an inner guide, the inner guide including an elongate body, the body of the inner guide being disposed within the hollow interior of the outer shaft.

### Example 9

The apparatus of Example 8, the body of the knife assembly being laterally offset relative to the body of the inner guide within the hollow interior of the shaft.

### Example 10

The apparatus of any of Examples 8 through 9, the body of the knife assembly being positioned adjacent to and in sliding contact with the body of the inner guide.

### Example 11

The apparatus of any of Examples 8 through 10, the body of the knife assembly having a length, the body of the knife assembly having a first thickness along a first region of the length of the body of the knife assembly, the body of the knife assembly having a second thickness along a second region of the length of the body of the knife assembly, the body of the inner guide having a length, the body of the inner guide having a first thickness along a first region of the length of the body of the inner guide, the body of the inner guide having a second thickness along a second region of the length of the body of the inner guide.

### Example 12

The apparatus of Example 11, the body of the knife assembly and the body of the inner guide being positioned to provide contact between: (i) the body of the knife assembly along the first region of the length of the body of the knife assembly and the body of the inner guide along the second region of the length of the body of the inner guide, and (ii) the body of the knife assembly along the second region of the length of the body of the knife assembly and the body of the inner guide along the second region of the length of the body of the inner guide.

### Example 13

The apparatus of Example 12, the first thickness of the body of the knife assembly being larger than the second thickness of the body of the knife assembly, the first thickness of the body of the inner guide being smaller than the second thickness of the body of the inner guide.

### Example 14

The apparatus of any of Examples 1 through 13, the elongate body further including a notch, the apparatus further including a knife firing assembly, the knife firing assembly being configured to engage the elongate body via the notch.

### Example 15

The apparatus of Example 14, the knife firing assembly including a clip having a protrusion disposed in the notch.

### Example 16

The apparatus of any of Examples 1 through 15, the end effector further comprising one or more electrodes, the one or more electrodes being operable to apply radiofrequency energy to tissue.

### Example 17

The apparatus of any of Examples 1 through 16, the body including a structure selected from the group consisting of a handle and a robotic system interface.

### Example 18

An apparatus comprising: (a) a body; (b) a shaft assembly extending distally relative to the body, the shaft assembly including: (i) an outer shaft defining a hollow interior and further defining a central longitudinal axis, (ii) a knife assembly including: (A) an elongate body having a proximal end and a distal end, at least a portion of the body being slidably positioned within the hollow interior of the outer shaft, the elongate body defining a central longitudinal axis, the central longitudinal axis of the elongate body being laterally offset from the central longitudinal axis of the outer shaft, and (B) a beam extending distally from the distal end of the body, the beam including a distal portion having a knife, the knife being operable to sever tissue, and (iii) an inner guide disposed within the hollow interior of the outer shaft, the inner guide defining a central longitudinal axis, the central longitudinal axis of the inner guide being laterally offset from the central longitudinal axis of the outer shaft, the central longitudinal axis of the inner guide also being laterally offset from the central longitudinal axis of the elongate body of the knife assembly; and (c) an end effector positioned at a distal end of the shaft assembly, the end effector including: (i) a first jaw, and (ii) a second jaw, the first jaw being movable relative to the second jaw between an open position and a closed position, the knife being operable to translate along one or both of the first jaw or the second jaw.

### Example 19

The apparatus of Example 19, the beam including a proximal portion positioned within the body and thereby secured to the body.

### Example 20

A method comprising: (a) forming a knife assembly, forming a knife assembly comprising: (i) overmolding an elongate body along a proximal portion of a beam, the elongate body defining a central longitudinal axis, and (ii) forming a blade at a distal end of the beam; (c) positioning an inner guide body in relation to the knife assembly, thereby forming a combination, the inner guide body defining a central longitudinal axis, the central longitudinal axis of the inner guide body being positioned laterally relative to the central longitudinal axis of the elongate body of the knife assembly; (d) positioning an outer tube along the combination of the knife assembly and the inner guide body, thereby forming a shaft assembly, the outer tube defining a central longitudinal axis, the central longitudinal axis of the inner guide body being laterally offset from the central longitudinal axis of the elongate body of the knife assembly, the central longitudinal axis of the inner guide body being laterally offset from the central longitudinal axis of the inner guide body; and (e) securing an end effector in relation to the shaft assembly, the end effector including: (i) a first jaw, and (ii) a second jaw, the first jaw being movable relative to the second jaw between an open position and a closed position, the knife being operable to translate along one or both of the first jaw or the second jaw.

### IV. Miscellaneous

Any of the versions of the instruments described herein may include various other features in addition to or in lieu of those described above. While the examples herein are described mainly in the context of electrosurgical instruments, various teachings herein may be readily applied to a variety of other types of devices. By way of example only, the various teachings herein may be readily applied to other types of electrosurgical instruments, tissue graspers, tissue retrieval pouch deploying instruments, surgical staplers, surgical clip appliers, ultrasonic surgical instruments, etc. The teachings herein may be readily applied to any of the instruments described in any of the references cited herein. Any of the devices herein may also include one or more of the various features disclosed in any of the various references that are incorporated by reference herein. The teachings herein may thus be readily combined with the teachings of any of the references cited herein in numerous ways. Other types of instruments into which the teachings herein may be incorporated will be apparent to those skilled in the art in view of the teachings herein.

It should be appreciated that any patent, publication, or other disclosure material, in whole or in part, that is said to be incorporated by reference herein is incorporated herein only to the extent that the incorporated material does not conflict with existing definitions or other disclosure material set forth in this disclosure. The disclosure as explicitly set forth herein supersedes any conflicting material incorporated herein by reference. Any material, or portion thereof, that is said to be incorporated by reference herein, but which conflicts with existing definitions or other disclosure material set forth herein will only be incorporated to the extent that no conflict arises between that incorporated material and the existing disclosure.

The terms “proximal” and “distal” are defined herein relative to a surgeon, robotic arm, or other operator or structure grasping a surgical instrument having a distal surgical end effector. The term “proximal” refers the position of an element closer to the surgeon, robotic arm, or other operator or structure; and the term “distal” refers to the position of an element closer to the surgical end effector of the surgical instrument and further away from the surgeon or other operator or structure.

As used herein, an element or step recited in the singular and proceeded with the word “a” or “an” should be understood as not excluding plural of said elements or steps, unless such exclusion is explicitly stated. Furthermore, references to “one example” or “an example” are not intended to be interpreted as excluding the existence of additional examples that also incorporate the recited features. The use of “including,” “comprising,” or “having” and variations thereof herein is meant to encompass the items listed thereafter, and equivalents thereof, as well as additional items.

As used herein in the specification and claims, including as used in the examples and unless otherwise expressly specified, the terms “about” or “approximately” for any numerical values or ranges indicate a suitable dimensional tolerance, or other form of reasonable expected range, that allows the part or collection of components to function for its intended purpose as described herein. More specifically, “about” or “approximately” may refer to the range of values that are within ±10% of the recited value (e.g., “about 100” may refer to the range of values from 90 to 110, including 90, 110, 100, and all other values within the range of 90 and 110). Any numerical values given herein should also be understood to include about or approximately that value unless the context indicates otherwise. For example, if the value “10” is disclosed, then “about 10” is also disclosed. Any numerical range recited herein is intended to include all sub-ranges subsumed therein. The terms “approximately” and “about” are thus utilized herein to represent the degree by which a quantitative representation may vary from a stated reference without resulting in a change in the basic function of the subject matter at issue.

The term “substantially” is also utilized herein to represent the degree by which a quantitative representation may vary from a stated reference without resulting in a change in the basic function of the subject matter at issue. The term “substantially” shall therefore be understood to include a range of conditions or results that provide a functional equivalent to an explicitly stated condition or result. For instance, if a task is “substantially complete,” the result of the task having been substantially completed is functionally equivalent to the result that would have been achieved if the task had been perfectly completed. As another non-limiting example, a component that is “substantially straight” or “substantially flat,” an apparatus including a component that is “substantially straight” or “substantially flat” may provide a result or effect that is functionally equivalent to a result or effect that would be achieved by the same apparatus including the same component in a perfectly straight or perfectly flat configuration. The range implied by the term “substantially” should also be read to include the perfect result that is within that range. Thus, the term “substantially complete” shall be read as including “perfectly complete” while also including a range of completeness that is functionally equivalent to perfectly complete. As another example, terms such as “substantially straight” and “substantially flat” shall be read as including “perfectly straight” and “perfectly flat,” respectively; while also including a range of straightness or flatness that is functionally equivalent to perfectly straight or flat, respectively. As with the terms “approximately” and “about,” the term “substantially” may indicate a suitable dimensional tolerance, or other form of reasonable expected range, that allows a part or collection of components to function for its intended purpose as described herein.

The limitations of the following claims are not written in means-plus-function format and are not intended to be interpreted based on 35 U.S.C. §112(f), unless such claim limitations expressly use the phrase “means for” followed by a statement of function void of further structure.

Versions described above may be designed to be disposed of after a single use, or they can be designed to be used multiple times. Versions may, in either or both cases, be reconditioned for reuse after at least one use. Reconditioning may include any combination of the steps of disassembly of the device, followed by cleaning or replacement of particular pieces, and subsequent reassembly. In particular, some versions of the device may be disassembled, and any number of the particular pieces or parts of the device may be selectively replaced or removed in any combination. Upon cleaning and/or replacement of particular parts, some versions of the device may be reassembled for subsequent use either at a reconditioning facility, or by an operator immediately prior to a procedure. Those skilled in the art will appreciate that reconditioning of a device may utilize a variety of techniques for disassembly, cleaning/replacement, and reassembly. Use of such techniques, and the resulting reconditioned device, are all within the scope of the present application.

By way of example only, versions described herein may be sterilized before and/or after a procedure. In one sterilization technique, the device is placed in a closed and sealed container, such as a plastic or TYVEK bag. The container and device may then be placed in a field of radiation that can penetrate the container, such as gamma radiation, x-rays, or high-energy electrons. The radiation may kill bacteria on the device and in the container. The sterilized device may then be stored in the sterile container for later use. A device may also be sterilized using any other technique known in the art, including but not limited to beta or gamma radiation, ethylene oxide, or steam.

What is claimed is:

1. An apparatus comprising:
(a) a body;
(b) a shaft assembly extending distally relative to the body, the shaft assembly including:
(i) an outer shaft defining a hollow interior, and
(ii) a knife assembly including:
(A) an elongate body having a proximal end and a distal end, at least a portion of the body being slidably positioned within the hollow interior of the outer shaft, and
(B) a beam extending distally from the distal end of the body, the beam including a proximal portion positioned within the body and thereby secured to the body, the beam further including a distal portion having a knife, the knife being operable to sever tissue; and
(c) an end effector positioned at a distal end of the shaft assembly, the end effector including:
(i) a first jaw, and
(ii) a second jaw, the first jaw being movable relative to the second jaw between an open position and a closed position, the knife being operable to translate along one or both of the first jaw or the second jaw.

2. The apparatus of claim 1, the body comprising a plastic material.

3. The apparatus of claim 2, the beam comprising a metal material.

4. The apparatus of claim 3, the plastic material in a distal portion of the body surrounding the metal material in the proximal portion of the beam as an overmold about the metal material.

5. The apparatus of claim 1, the body having a length, the body having a first thickness along a first region of the length of the body, the body having a second thickness along a second region of the length of the body, the proximal portion of the beam being disposed within the second region of the length of the body.

6. The apparatus of claim 1, the proximal portion of the beam including a notch, the notch being configured to promote a rigid coupling between the beam and the body.

7. The apparatus of claim 1, the outer shaft defining a central longitudinal axis, the body of the knife assembly defining a central longitudinal axis, the central longitudinal axis of the knife assembly being laterally offset relative to the central longitudinal axis of the outer shaft.

8. The apparatus of claim 1, the shaft assembly further including an inner guide, the inner guide including an elongate body, the body of the inner guide being disposed within the hollow interior of the outer shaft.

9. The apparatus of claim 8, the body of the knife assembly being laterally offset relative to the body of the inner guide within the hollow interior of the shaft.

10. The apparatus of claim 8, the body of the knife assembly being positioned adjacent to and in sliding contact with the body of the inner guide.

11. The apparatus of claim 8, the body of the knife assembly having a length, the body of the knife assembly having a first thickness along a first region of the length of the body of the knife assembly, the body of the knife assembly having a second thickness along a second region of the length of the body of the knife assembly,
the body of the inner guide having a length, the body of the inner guide having a first thickness along a first region of the length of the body of the inner guide, the body of the inner guide having a second thickness along a second region of the length of the body of the inner guide.

12. The apparatus of claim 11, the body of the knife assembly and the body of the inner guide being positioned to provide contact between:
(i) the body of the knife assembly along the first region of the length of the body of the knife assembly and the body of the inner guide along the second region of the length of the body of the inner guide, and
(ii) the body of the knife assembly along the second region of the length of the body of the knife assembly and the body of the inner guide along the second region of the length of the body of the inner guide.

13. The apparatus of claim 12, the first thickness of the body of the knife assembly being larger than the second thickness of the body of the knife assembly, the first thickness of the body of the inner guide being smaller than the second thickness of the body of the inner guide.

14. The apparatus of claim 1, the elongate body further including a notch, the apparatus further including a knife firing assembly, the knife firing assembly being configured to engage the elongate body via the notch.

15. The apparatus of claim 14, the knife firing assembly including a clip having a protrusion disposed in the notch.

16. The apparatus of claim 1, the end effector further comprising one or more electrodes, the one or more electrodes being operable to apply radiofrequency energy to tissue.

17. The apparatus of claim 1, the body including a structure selected from the group consisting of a handle and a robotic system interface.

18. An apparatus comprising:
(a) a body;
(b) a shaft assembly extending distally relative to the body, the shaft assembly including:
(i) an outer shaft defining a hollow interior and further defining a central longitudinal axis,
(ii) a knife assembly including:
(A) an elongate body having a proximal end and a distal end, at least a portion of the body being slidably positioned within the hollow interior of the outer shaft, the elongate body defining a central longitudinal axis, the central longitudinal axis of the elongate body being laterally offset from the central longitudinal axis of the outer shaft, and
(B) a beam extending distally from the distal end of the body, the beam including a distal portion having a knife, the knife being operable to sever tissue, and
(iii) an inner guide disposed within the hollow interior of the outer shaft, the inner guide defining a central longitudinal axis, the central longitudinal axis of the inner guide being laterally offset from the central longitudinal axis of the outer shaft, the central longitudinal axis of the inner guide also being laterally offset from the central longitudinal axis of the elongate body of the knife assembly; and
(c) an end effector positioned at a distal end of the shaft assembly, the end effector including:
(i) a first jaw, and
(ii) a second jaw, the first jaw being movable relative to the second jaw between an open position and a closed position, the knife being operable to translate along one or both of the first jaw or the second jaw.

19. The apparatus of claim 18, the beam including a proximal portion positioned within the body and thereby secured to the body.

20. A method comprising:
(a) forming a knife assembly, forming a knife assembly comprising:
(i) overmolding an elongate body along a proximal portion of a beam, the elongate body defining a central longitudinal axis, and
(ii) forming a blade at a distal end of the beam;
(c) positioning an inner guide body in relation to the knife assembly, thereby forming a combination, the inner guide body defining a central longitudinal axis, the central longitudinal axis of the inner guide body being positioned laterally relative to the central longitudinal axis of the elongate body of the knife assembly;
(d) positioning an outer tube along the combination of the knife assembly and the inner guide body, thereby forming a shaft assembly, the outer tube defining a central longitudinal axis, the central longitudinal axis of the inner guide body being laterally offset from the central longitudinal axis of the elongate body of the knife assembly, the central longitudinal axis of the inner guide body being laterally offset from the central longitudinal axis of the inner guide body; and
(e) securing an end effector in relation to the shaft assembly, the end effector including:
(i) a first jaw, and
(ii) a second jaw, the first jaw being movable relative to the second jaw between an open position and a closed position, the knife being operable to translate along one or both of the first jaw or the second jaw.
